(19) United States
(12) Patent Application Publication
(10) Pub. No.: US 2016/0349336 A1
(43) Pub. Date: Dec. 1, 2016

(54) HEAD COIL AND MAGNETIC RESONANCE IMAGING APPARATUS EMPLOYING THE SAME

(71) Applicant: Samsung Electronics Co., Ltd., Gyeonggi-do (KR)

(72) Inventors: Min-soo CHANG, Gyeonggi-do (KR); Seul-gi PARK, Gyeonggi-do (KR); Ju-hyung LEE, Gyeonggi-do (KR)

(21) Appl. No.: 15/078,243

(22) Filed: Mar. 23, 2016

(30) Foreign Application Priority Data
May 28, 2015 (KR) 10-2015-0075366

(51) Int. Cl. G01R 33/341; A61B 5/055

(52) U.S. Cl. CPC G01R 33/341; A61B 5/055

(57) ABSTRACT

A head coil and a magnetic resonance imaging (MRI) apparatus employing the same. The head coil may increase a field of view by allowing a housing over the eyes of a patient to be closely fitted around the head of the patient. The head coil includes a plurality of radio frequency (RF) coil elements, a first housing, and a second housing configured to receive the head of a patient along with the first housing, wherein the first and second housings are changed or modified to have any of receiving spaces of different sizes to correspond to any of different head sizes of patients.

### CLAIM OF PRIORITY

This application claims the benefit of priority from Korean Patent Application No. 10-2015-0075366, filed on May 28, 2015, in the Korean Intellectual Property Office, the disclosure of which is incorporated herein in its entirety by reference.

### BACKGROUND

1. Field of the Disclosure

The present disclosure relates to head coils and magnetic resonance imaging (MRI) apparatuses employing the same. More particularly, the present disclosure is related to a head coil having a housing structure corresponding to a size of the head of a patient and an MRI apparatus employing the head coil.

2. Description of the Related Art

A radio frequency (RF) coil that acts as a detector for receiving a weak RF signal output from a patient in a process of obtaining an image in a magnetic resonance imaging (MRI) apparatus performs a function that is important to provide an accurate image. Various types of RF coils are required according to body parts whose images are to be obtained (e.g., head, spine, shoulders, breast, torso, knees, and ankles).

A head coil, which is a kind of the RF coil, is used to obtain a medical image of the head of a patient. The head coil is a coil has a space which receives a patient's head, and an image is captured after the head is placed in the head coil. A plurality of RF coil elements are arranged in a housing of the head coil, and an image is obtained by arranging and synthesizing signals respectively received from the plurality of RF coil elements.

RF coil elements of a conventional head coil are designed based on a fixed size housing. However, sizes (volumes) of heads vary according to patients. Accordingly, a distance between RF coil elements and the head of a patient varies according to a size of the head, an intensity of each of received signals varies, and thus the quality of an obtained image varies according to the patient. For example, when an image is to be obtained of a patient having a head size (volume) that was expected when a housing of a head coil was designed, since a plurality of RF coil elements of the head coil have optimal matching and tuning impedances, the intensities of signals respectively received from the plurality of RF coil elements are the same, thereby making it possible to obtain a high-quality image. However, when an image is to be obtained of a patient having a head size (volume) that is less or greater than the expected size (volume), since a distance between the head and a plurality of RF coil elements is too large or too small and matching and tuning impedances vary according to the plurality of RF coil elements, intensities of signals respectively received from the plurality of RF coil devices are different from one another, thereby making it difficult to obtain a high-quality image. Also, when imaging is performed for a long time, since a hard housing structure has a small field of view, a patient may feel enclosed and tired.

### SUMMARY

The disclosure provides a head coil structure including one or more head coils that may obtain a high-quality image by using a housing structure corresponding to a size of the head of a patient and magnetic resonance imaging (MRI) apparatuses employing the head coils.

The disclosure also provides a head coil structure including one or more head coils that may increase a field of view by allowing a housing over the eyes of a patient to be closely fitted around the head of the patient and MRI apparatuses employing the head coils.

Additional aspects will be set forth in part by the disclosure which follows and, in part, will become more apparent to a person of ordinary skill in the art, or may be learned by practice of the presented embodiments.

According to an aspect of the disclosure, a head coil for a magnetic resonance imaging (MRI) apparatus includes: a plurality of radio frequency (RF) coil elements; a first housing having a first housing surface in which one or more of the plurality of RF coil elements is embedded therein, and the first housing having an adjustable receiving space that corresponds to any of different heads sizes of patients; a second housing having a structure configured to receive the head of a patient along with the first housing and having a second housing surface in which another one or more of the plurality of RF coil elements is embedded therein, and having one receiving space selected from among a plurality of second housings with respective receiving spaces of different sizes that correspond to different head sizes of patients; and a coupling unit that detachably couples to any one of the plurality of the second housings to the first housing. The first housing may be detachably mounted on a structure (e.g. a support) that supports the patient. The first housing may cover the back part of the head of the patient, and the second housing may cover at least a part of the front part of the head of the patient.

The first housing may include a plurality of housing pieces, and a fixing member configured to fix the plurality of housing pieces and to vary the receiving space by adjusting a fixed position between the plurality of housing pieces.

A shielding member for shielding electromagnetic coupling between adjacent RF coil elements may be provided on each of the plurality of RF coil elements. Each of the plurality of RF coil elements may be an RF loop coil, and the shielding member may be a shield loop coil surrounding an outer surface of the RF loop coil. The shield loop coil may be located on the same plane as a plane on which the RF loop coil is located. The shield loop coil was arranged to have the same center as a center of the RF loop coil.

According to an aspect of the disclosure, a head coil structure for a magnetic resonance imaging (MRI) apparatus includes: a plurality of radio frequency (RF) coil elements; a fixed housing unit having a structure in which at least one or more of a plurality of RF coil elements are embedded therein to cover a back part of a head of a patient; an adjustable-size housing unit configured to receive the head of the patient along with the fixed housing unit, in which the adjustable-size housing unit having embedded therein another one or more of the plurality of RF coil elements, and comprising a plurality of housing pieces that extend from a plurality of points of a circumference of the fixed housing unit and are bendably connected to one another; and a fixing member configured to detachably fix a first and a second housing piece of the plurality of housing pieces respectively located at ends of the fixing member extending from a first point and a second point of the circumference of the fixed housing unit. The housing unit may be variable (i.e. adjustably-sized) to receive the head of the patient along with the fixed housing unit and to embed therein some of the plurality of RF coil elements.

The plurality of housing pieces may be connected to one another by using hinges.

The fixing member may vary a size of a receiving space of the head of the patient formed by the fixed housing unit and the adjustable-size housing unit by adjusting a fixed position between the first distal housing piece and the second distal housing piece to correspond to any of different head sizes of patients. The first distal housing piece and the second distal housing piece may be coupled to each other to partially overlap each other.

The adjustable-size housing unit may include an eye housing piece located over the eyes of the patient and a mouth housing piece located over the mouth of the patient, and the eye housing piece and the mouth housing piece may have openings through which the eyes and the mouth of the patient are exposed to the outside. The eye housing piece and the mouth housing piece respectively may include an eye RF coil and a mouth RF coil, and each of the eye RF coil and the mouth RF coil may be a circular, elliptical, or polygonal loop coil.

A shielding member for shielding electromagnetic coupling between adjacent RF coil elements may be provided on each of the plurality of RF coil elements. Each of the plurality of RF coil elements may be comprised of an RF loop coil, and the shielding member may be comprised of a shield loop coil surrounding an outer surface of the RF loop coil. The shield loop coil may be located on the same plane as a plane on which the RF loop coil is located. The shield loop coil may be arranged to have the same center as a center of the RF loop coil.

As an inner receiving space of the adjustable-size housing unit is changed, a distance between at least some of the plurality of RF coil elements in the adjustable-size housing unit may be changed or an amount by which at least some of the plurality of RF coil elements overlap each other may be changed.

According to another embodiment of the disclosure, a magnetic resonance imaging (MRI) apparatus may include: a gantry including an imaging space to which a magnetic field for obtaining a magnetic resonance (MR) image is applied; a main magnet module arranged in the gantry and configured to apply a main magnetic field; a gradient coil module provided in the gantry and configured to apply a gradient magnetic field; a main body radio frequency (RF) coil module provided in the gantry; and a head coil configured to cover at least a part of the head of a patient, wherein the head coil includes: a plurality of RF coil elements; a first housing configured to embed therein some of the plurality of RF coil elements and having a receiving space that varies to correspond to any of different head sizes of patients; a second housing configured to receive the head of the patient along with the first housing and to embed therein others of the plurality of RF coil elements, and having one receiving space from among receiving spaces of different sizes to correspond to any of different head sizes of patients; and a coupling unit configured to detachably couple any one of a plurality of the second housings having receiving spaces of different sizes to the first housing.

According to an aspect of another embodiment, a magnetic resonance imaging (MRI) apparatus includes: a gantry including an imaging space to which a magnetic field for obtaining a magnetic resonance (MR) image is applied; a main magnet module provided in the gantry and configured to apply a main magnetic field; a gradient coil module provided in the gantry and configured to apply a gradient magnetic field; a main body radio frequency (RF) coil module provided in the gantry; and a head coil, wherein the head coil includes: a plurality of RF coil elements; a fixed housing unit configured to embed therein one or more of the plurality of RF coil elements and to cover the back part of the head of a patient; an adjustable-size housing unit configured to receive the head of the patient along with the fixed housing unit and to embed therein another or more of the plurality of RF coil elements, and including a plurality of housing pieces that extend from a plurality of points of a circumference of the fixed housing unit and are bendably connected to one another; and a fixing member configured to detachably fix first and second housing pieces respectively located at ends of a plurality of first and second housing pieces respectively extending from a first point and a second point of the circumference of the fixed housing unit.

Since a head coil allows RF coil elements to be closely (snugly) fitted around the head of each patient irrespective of a size (volume) of the head by changing or modifying a part of a housing, an MRI apparatus may obtain a high-quality image than with devices known heretofore.

Since the head coil may increase a field of view, even when imaging is performed for a long time, the extent to which the patient feels enclosed and tired may be minimized more than known heretofore.

### BRIEF DESCRIPTION OF THE DRAWINGS

These and/or other aspects of the disclosure will become more apparent and more readily appreciated by a person of ordinary skill in the art from the following description of the embodiments, taken in conjunction with the accompanying drawings in which:

FIG. 1 is a perspective view illustrating a state in which a head coil is mounted on a patient according to an embodiment;

FIG. 2 is a front view of the head coil of FIG. 1;

FIG. 3 is a perspective view of a first housing of the head coil of FIG. 1;

FIG. 4A and FIG. 4B are views illustrating an example in which a fixing member of the first housing of FIG. 3 is fixed;

FIG. 5 is a perspective view of a second housing of the head coil of FIG. 1;

FIG. 6A, FIG. 6B and FIG. 6C are front views illustrating examples of the second housing of FIG. 5 having different sizes;

FIG. 7 is a view illustrating a state in which a head coil is mounted on the patient according to another embodiment;

FIG. 8 is a view illustrating a state in which the head coil of FIG. 7 is unfolded;

FIG. 9A and FIG. 9B are views illustrating a process of mounting the head coil of FIG. 8 on the head of the patient;

FIG. 10A and FIG. 10B are views illustrating an example in which the head coil of FIG. 8 is fixed by using a fixing member;

FIG. 11A is a plan view of a radio frequency (RF) coil element of a head coil according to another embodiment;

FIG. 11B is a cross-sectional view of the RF coil element of FIG. 11A;

FIG. 12 is a plan view illustrating a state in which two RF coil elements overlap each other by a predetermined distance; and

FIG. 13 is a view of a magnetic resonance imaging (MRI) apparatus according to another embodiment.

### DETAILED DESCRIPTION

The advantages and features of the present disclosure and methods of achieving the advantages and features will be described more fully with reference to the accompanying drawings, in which embodiments of the present disclosure are shown. The disclosure, which may be embodied in many different forms, nevertheless should not be construed as being limited to the embodiments set forth herein; rather these embodiments are provided so that this disclosure will be thorough and complete, and will fully permit practice of the appended claims by one of ordinary skill in the art. Like reference numerals denote like elements in the drawings. In the attached drawings, sizes of elements may be exaggerated for clarity.

The terms used herein will be briefly explained and the present disclosure will be explained in detail.

Most of the terms used herein are general terms that have been widely used in the technical art to which the present disclosure pertains. However, some of the terms used herein may be created reflecting intentions of technicians in this art, precedents, or new technologies. Accordingly, the specific terms used herein should be understood based on the unique meanings thereof and the whole context of the present disclosure.

It will be further understood that when a part “includes” or “comprises” an element, unless otherwise defined, the part may further include other elements, not excluding the other elements.

The present disclosure will now be described more fully with reference to the accompanying drawings for those of ordinary skill in the art to be able to perform embodiments without any difficulty. Also, parts in the drawings unrelated to the detailed description are omitted to ensure clarity of the present disclosure.

A magnetic resonance imaging (MRI) apparatus is an apparatus for obtaining a tomographic image of a part of an object by expressing, in a contrast image, a strength of a magnetic resonance (MR) signal with respect to a radio frequency (RF) signal generated in a magnetic field having a specific strength. For example, when a patient is placed in a strong magnetic field and an RF signal for resonating a specific atomic nucleus (e.g., a hydrogen atomic nucleus) is applied for an instant onto the patient and then is stopped, an MR signal is emitted from the specific atomic nucleus, and thus an MRI apparatus may receive the MR signal and may obtain an MR image. The MR signal is an RF signal emitted from the object. An intensity of the MR signal may be determined according to the concentration of predetermined atoms (e.g., hydrogen) included in the object, a relaxation time T1, a relaxation time T2, and a blood flow.

MRI apparatuses have characteristics different from those of other imaging apparatuses. Unlike imaging apparatuses such as computed tomography (CT) apparatuses that obtain images dependent upon a direction of detection hardware, MRI apparatuses may obtain two-dimensional (2D) images or three-dimensional (3D) volume images that are oriented toward an optional point. MRI apparatuses do not expose radiation to objects and examinees, unlike CT apparatuses, X-ray apparatuses, position emission tomography (PET) apparatuses, and single photon emission CT (SPECT) apparatuses, may obtain images having high soft tissue contrast, and may acquire neurological images, intravascular images, musculoskeletal images, and oncologic images in which it is important to precisely depict abnormal tissues.

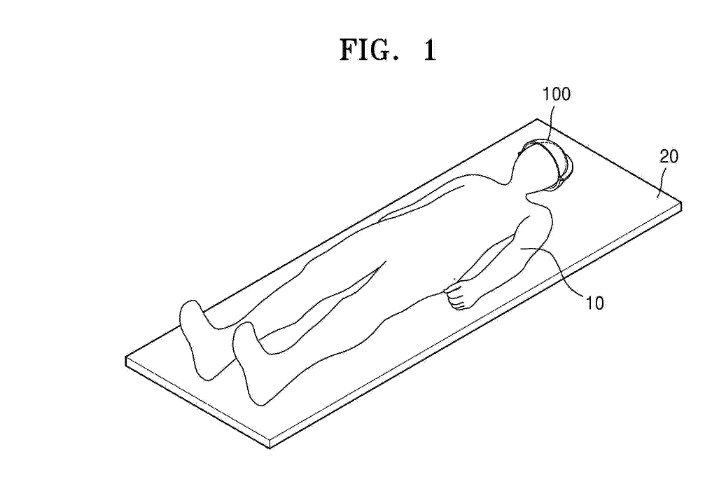

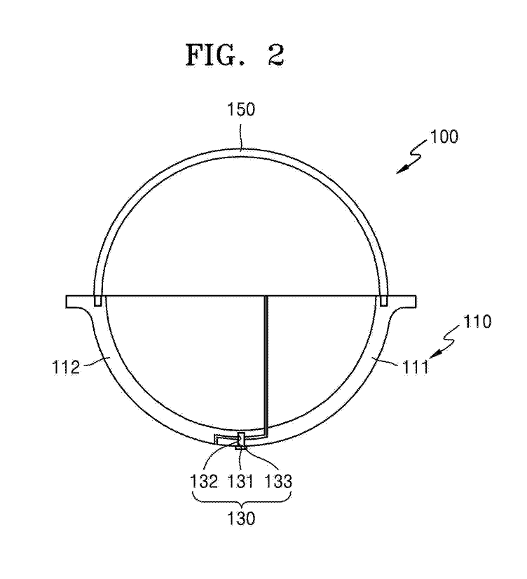

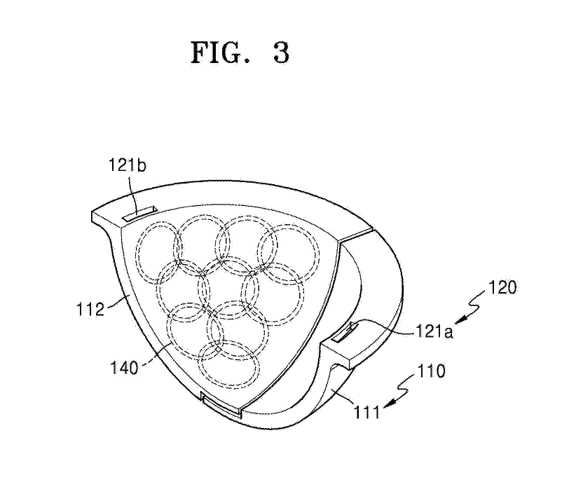

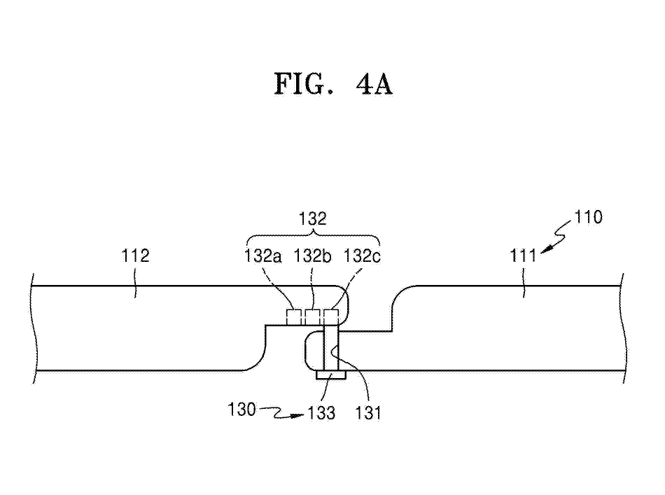

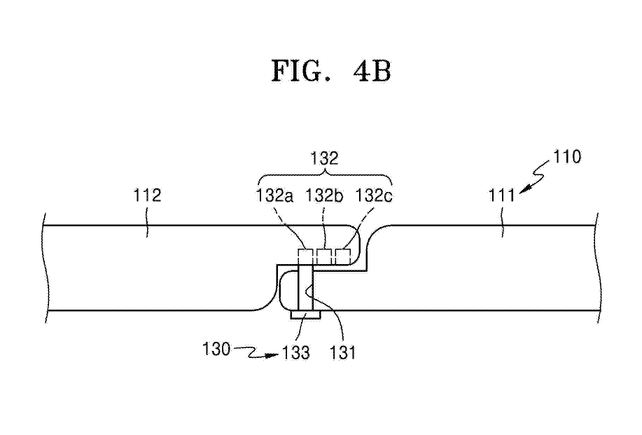

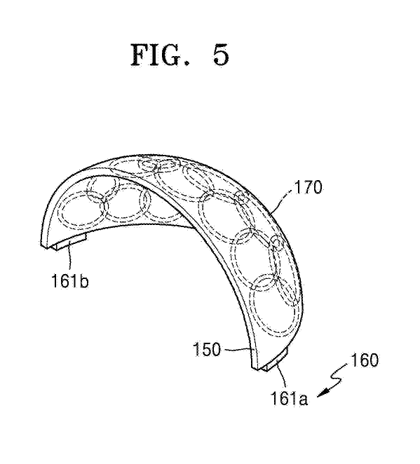

FIG. 1 is a perspective view illustrating a state in which a head coil 100 is mounted on a patient 10 according to an embodiment. FIG. 2 is a front view of the head coil 100. FIG. 3 is a perspective view of a first housing 110 of the head coil 100. FIGS. 4A and 4B are views illustrating an example in which a fixing member of the first housing 110 is fixed. FIG. 5 is a perspective view of a second housing 150 of the head coil 100.

Referring to FIGS. 1 through 5, as shown in FIG. 2 the head coil 100 includes the first housing 110, the second housing 150, and a plurality of RF coil elements 140 and 170. The first housing 110 and the second housing 150 are coupled to each other to provide a receiving space having a hemispherical shape in which the head of the patient 10 may be received.

The first housing 110 includes a first housing piece 111 and a second housing piece 112, and covers the back part of the head of the patient 10. For example, each of the first and second housing pieces 111 and 112 may be shaped as about a quarter of a full sphere, and when the first and second housing pieces 111 and 112 are coupled to each other, the first and second housing pieces 111 and 112 may have a hemispherical shape. The first and second housing pieces 111 and 112 may be formed of a rigid material or a flexible material. Also, the first housing 110 includes the plurality of RF coil elements 140, and includes a first coupling unit 120 (see FIG. 3) for coupling the first housing 110 and the second housing 150. Although the plurality of RF coil elements 140 are provided only in the second housing piece 112 of the first housing 110 in FIG. 3, the plurality of RF coil elements 140 are also provided in the first housing piece 111.

The first housing 110 may include a fixing member 130 configured to adjust a fixed position between the first and second housing pieces 111 and 112 in order to vary the receiving space. Fixed portions of the first and second housing pieces 111 and 112 may be stepped so that the fixed portions of the first and second housing pieces 111 and 112 may overlap each other, and thus thicknesses of the fixed portions may be small.

With references to FIGS. 4A and 4B, the fixing member 130 may include a first fixing sub-member 131 provided on the fixed portion of the first housing piece 111, and a second fixing sub-member 132 provided on the fixed portion of the second housing piece 112. The first fixing sub-member 131 may be a hole into which a bolt 133 may be inserted, and the second fixing sub-member 132 may be a female screw engaged with the bolt 133. As further shown in FIGS. 4A and 4B, a plurality of female screw holes 132a, 132b, and 132c of the second fixing sub-member 132 may be formed to correspond to a size of the head. Alternatively, a plurality of holes of the first fixing sub-member 131 may be formed to correspond to a size of the head. The above structure of the fixing member 130 is and the present embodiment is not limited thereto. For example, the first fixing sub-member 131 may be formed as a long slot that extends in one direction, and the second fixing sub-member 132 may include one female screw hole. Alternatively, the fixing member 130 may have a structure in which a protrusion is inserted into a groove or any of other well-known fasteners.

The bolt 133 may be inserted into one of the female screw holes 132a, 132b, and 132c according to an amount by which the first and second housing pieces 111 and 112 overlap each other. For example, when the head of the patient 10 is large, since the first and second housing pieces 111 and 112 overlap each other by a small amount, the bolt 133 is inserted into the female screw hole 132c that is an outermost hole from among the female screw holes 132a, 132b, and 132c as shown in FIG. 4A. When the head of the patient 10 is small, since the first and second housing pieces 111 and 112 overlap each other by a large amount, the bolt 133 is inserted into the female screw hole 132a that is an innermost hole from among the female screw holes 132a, 132b, and 132c as shown in FIG. 4B. Although three female screw holes 132a, 1332b, and 132c are shown in FIGS. 4A and 4B, the present embodiment showing the fixing member is not limited thereto and, for example, two female screw holes may be formed or four or more female screw holes may be formed. With each screen hole comes another size that the head coil may fit on.

Although the first housing 110 has a hemispherical shape in FIG. 3, the present embodiment is not limited thereto. The first housing 110 may extend to the neck of the patient 10. Alternatively, the first housing 110 may have a shape corresponding to a shape of the back part of the head of a human being.

With reference to FIG. 1, the first housing 110 may be detachably mounted on a table 20 on which the patient 10 lies by using a coupling device (not shown). For example, after the first housing 110 is installed on the table 20, the patient 10 lies on the table 20. The receiving space of the first housing 110 is adjusted before or after the patient 10 lies on the table 20. Next, in a state where the patient 10 lies on the table 20, the second housing 150 may be coupled to the first housing 110.

The second housing 150 includes the plurality of RF coil elements 170 and a second coupling unit 160 for coupling the second housing 150 and the first housing 110 together, and covers the front part of the head of the patient 10. Although the second housing 150 has a hemispherical shape in FIG. 5, the present embodiment is not limited thereto. The second housing 150 may cover the entire front part of the head of the patient 10 or may cover a portion other than the face of the front part of the head. Alternatively, the second housing 150 may be formed to correspond to a shape of the front part (including the face) of the head. When the second housing 150 covers the entire front part of the head of the patient 10, an opening may be formed over the eyes to expose the eyes of the face to the outside in order to minimize the extent to which the patient 10 feels enclosed and tired, and furthermore, an opening may be formed over the mouth to expose the mouth of the face to the outside. The second housing 150 may be formed of a rigid material or a flexible material.

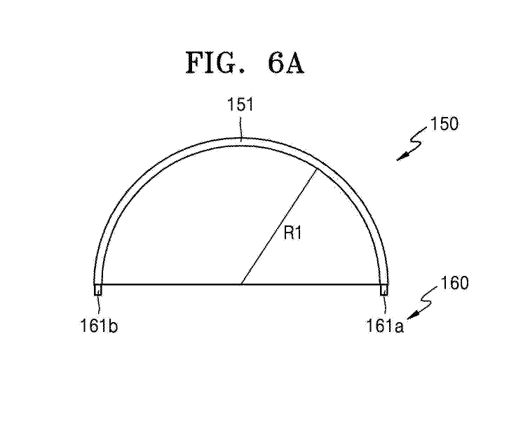

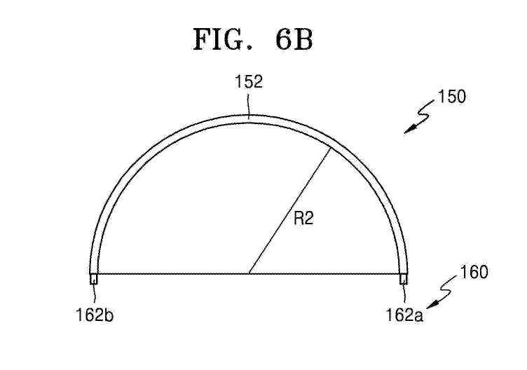

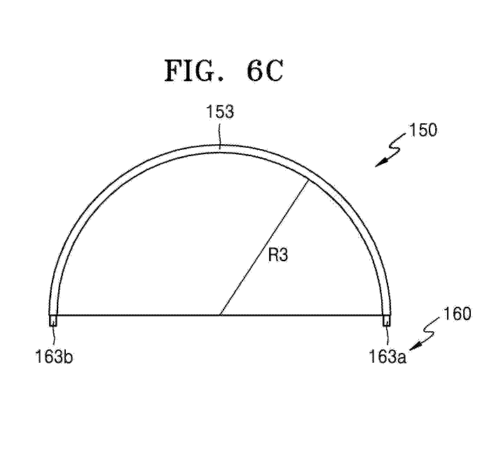

Sizes of heads may vary according to the age, and body size of patients, as the head is normally is some proportion to the body. For example, heads of adult men may be slightly larger than heads of adult women. For example, a size of the head of the patient 10, for example, a distance between ends of both ears, may range from about 132 mm to about 169 mm and a distance between the forehead and the back of the head may range from about 157 mm to about 216 mm. Accordingly, a plurality of the second housings 150 may be provided, and may have different receiving spaces. FIGS. 6A through 6C are front views illustrating examples of the second housing 150 having different sizes. For example, since an inner space of the second housing 150 may have substantially a hemispherical shape, a size of the second housing 150 may be roughly expressed by using a radius. For example, the second housing 150 may be a small second housing 151 having a size R1, a medium second housing 152 having a size R2, or a large second housing 153 having a size R3, wherein the sizes R1, R2, and R3 satisfy a relationship of R1<R2<R3. Although there are three sizes, the present embodiment is not limited thereto and the second housing 150 may have any of two sizes, or any of four or more sizes.

A coupling unit may include the first coupling unit 120 provided on the first housing 110 and the second coupling unit 160 provided on the second housing 150. For example, the first coupling unit 120 may include first and second grooves 121a and 122a respectively formed in two end portions of the first housing 110 that contact the second housing 150. The second coupling unit 160 may include first and second protrusions 161a and 161b formed on two end portions of the second housing 150 that contact the first housing 110. As described above, as a size of the second housing 150 is changed, a position of the second coupling unit 160 may also be changed. That is, as shown in FIGS. 6A through 6C, the first and second protrusions 161a and 161b of the small second housing 151, first and second protrusions 162a and 162b of the medium second housing 152, and first and second protrusions 163a and 163b of the large second housing 153 may be located at different positions in a diameter direction.

For example, when the head has a small size, the small second housing 151 is selected and is coupled to the first housing 110. For example, the first and second protrusions 161a and 161b of the small second housing 151 are fixedly inserted into the first and second grooves 121a and 121b of the first housing 110. In this case, since an interval between the first and second protrusions 161a and 161b of the small second housing 151 is relatively small, a fixed position between the first and second housing pieces 111 and 112 is adjusted so that an interval between the first and second grooves 121a and 121b of the first housing 110 corresponds to the interval between the first and second protrusions 161a and 161b. The adjustment of the fixed position between the first and second housing pieces 111 and 112 may be performed before the head of the patient 10 is placed in the first housing 110.

For example, when the head has a medium size, the medium second housing 152 is selected and is coupled to the first housing 110. In this case, since an interval between the first and second protrusions 162a and 162b of the medium second housing 152 is medium, a fixed position between the first and second housing pieces 111 and 112 is adjusted so that an interval between the first and second grooves 121a and 121b of the first housing 110 corresponds to the interval between the first and second protrusions 162a and 162b.

For example, when the head has a relatively large size, the large second housing 153 is selected and is coupled to the first housing 110. In this case, since an interval between the first and second protrusions 163a and 163b of the large second housing 153 is relatively large, a fixed position between the first and second housing pieces 111 and 112 is adjusted so that an interval between the first and second grooves 121a and 121b of the first housing 110 corresponds to the interval between the first and second protrusions 163a and 163b.

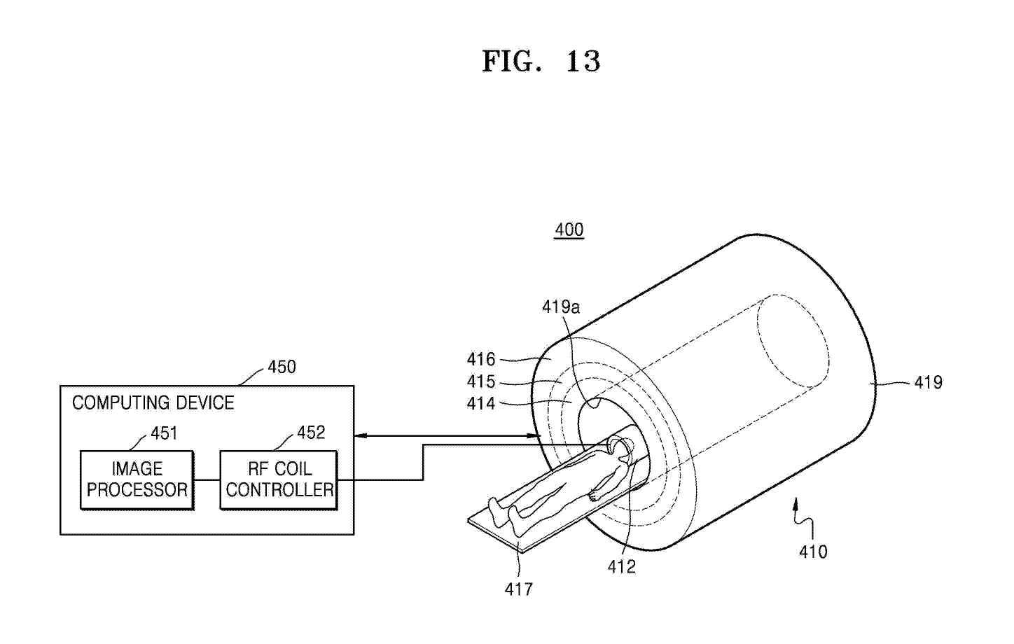

Since the receiving space of the first housing 110 is adjusted according to a size (volume) of the head of the patient 10 and the second housing 150 having an optimal size is selected, the RF coil elements 140 and 170 may be closely fitted around the head of the patient 10 and an MRI apparatus 400 (see FIG. 13) may obtain a high-quality image. Also, when an additional accessory such as a headset is worn on the head of the patient 10, the head coil 100 may secure a space for the accessory.

Electrical connection terminals (not shown) for transmitting signals received from the RF coil elements 170 in the second housing 150 may be provided on the first and second coupling units 120 and 160. Alternatively, a cable (not shown) for transmitting signals received from the RF coil elements 170 in the second housing 150 may be additionally provided.

The above structure of the coupling unit is and the present embodiment is not limited thereto. For example, the first and second coupling units 120 and 160 may have a structure in which a bolt screws into a nut or any of other well-known fasteners.

With reference to FIGS. 3 and 5, each of the plurality of RF coil elements 140 and 170 provided in the first and second housings 110 and 150 may be, for example, a circular, elliptical, or polygonal loop coil. The RF coil elements 140 and 170 are mounted in the first and second housings 110 and 150. The RF coil elements 140 and 170 may be arranged so that some overlap each other or are spaced apart from each other. A tuning capacitor, a matching capacitor, or a decoupling capacitor may be provided on the RF coil elements 140 and 170. The RF coil elements 140 and 170 may operate using a multi-channel method in which RF signals are independently input or output by an RF coil controller 452 (see FIG. 13). For example, in the RF coil elements 140 and 170, circuits may be arranged in parallel and intensities and phases of RF signals that are input or output may be independently controlled. If necessary, the RF coil elements 140 and 170 may be grouped and RF signals may be independently input or output according to groups. RF signals respectively received from the RF coil elements 140 and 170 may be arranged and synthesized to have a high reception sensitivity, thereby making it possible to obtain a high-quality image.

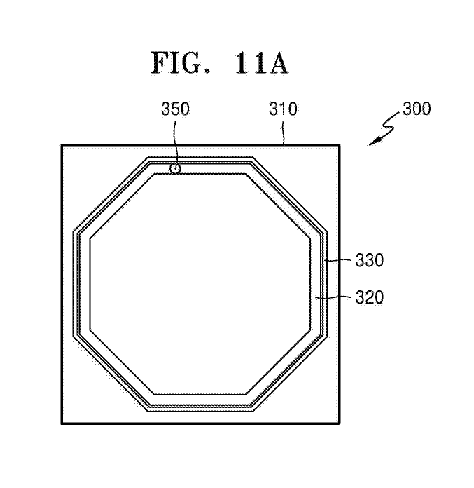

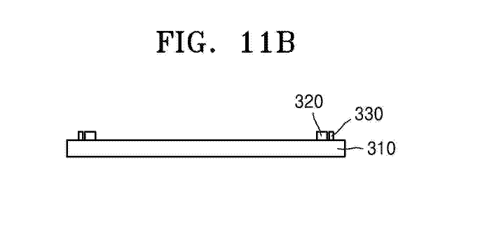

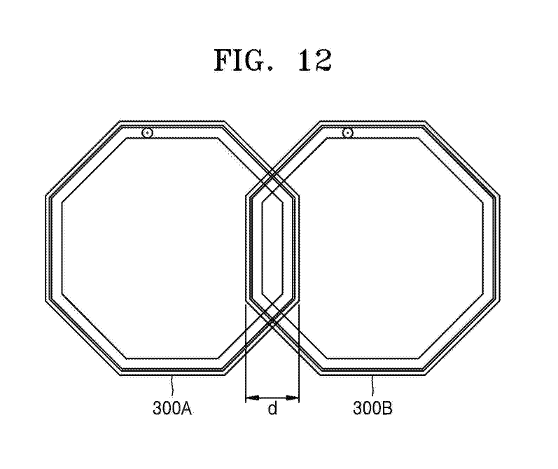

As described above with reference to FIG. 2, a fixed position between the first and second housing pieces 111 and 112 is adjusted in order to change the receiving space of the first housing 110. Accordingly, the fixed portions of the first and second housing pieces 111 and 112 may overlap each other (see FIGS. 4A and 4B), and an interval between the RF coil elements 140 in the first housing piece 111 and the RF coil elements 140 in the second housing piece 112 may be changed, and thus preset values such as a tuning impedance and a decoupling impedance may be changed. Accordingly, a shielding member 330 (see FIGS. 11A and 11B) for shielding electromagnetic coupling between adjacent RF coil elements may be disposed on each of the RF coil elements 140 and 170. The shielding member 330 will be explained below in detail with reference to FIGS. 11A, 11B, and 12.

As described above, a size of a receiving space for the head of each patient that is formed by the first and second housings 110 and 150 may be changed by adjusting a fixed position between the first and second housing pieces 111 and 112 of the first housing 110 according to a size of the head of the patient 10. Since a size of the first housing 110 may be adjusted according to a size of the head of each patient and the second housing 150 having an appropriate size may be selected, the head coil 100 including the first and second housings 110 and 150 may be understood as a reconfigurable head coil.

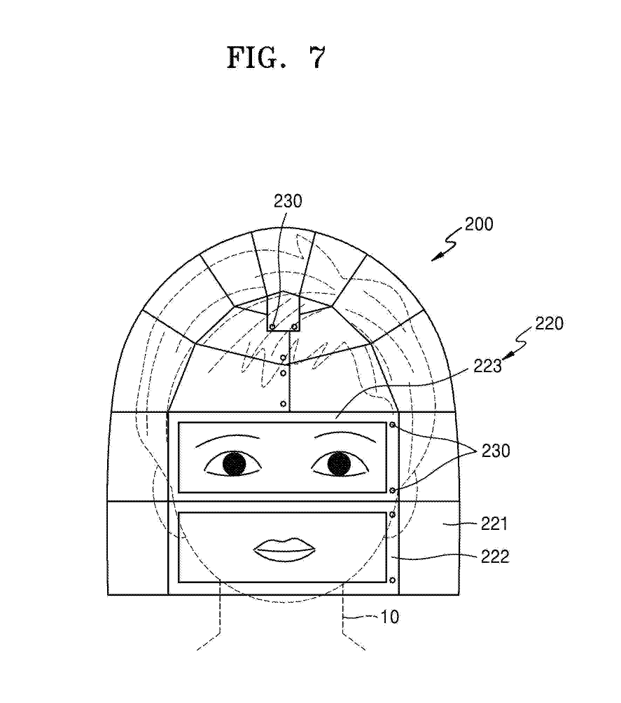

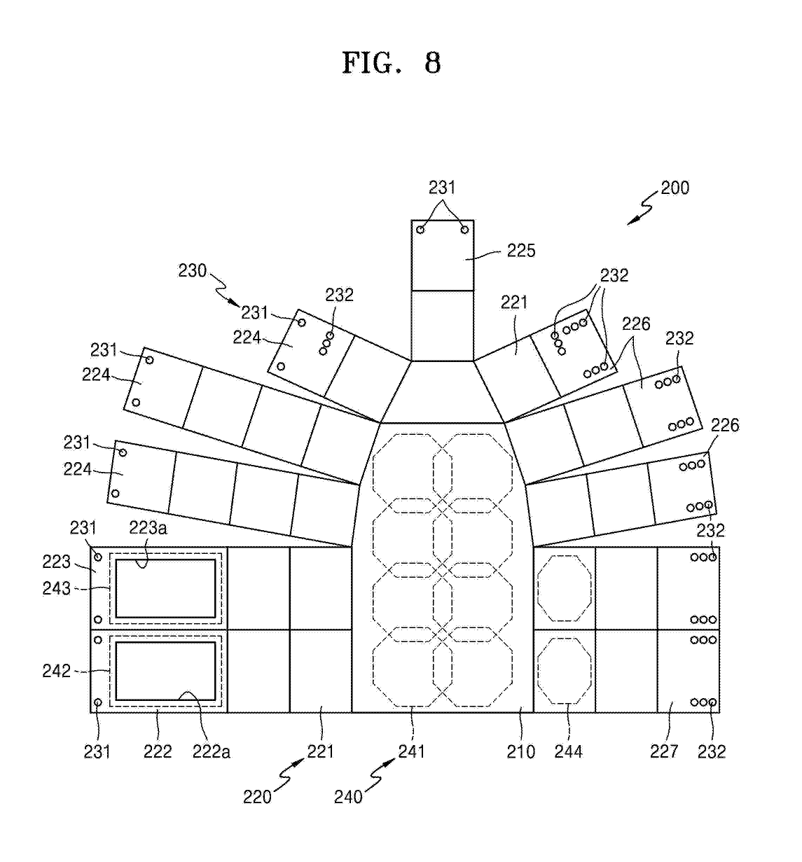

FIG. 7 is a view illustrating a state in which a head coil 200 is mounted on the patient 10 according to another embodiment of the present disclosure. FIG. 8 is a view illustrating a state in which the head coil 200 is unfolded.

Referring now to FIGS. 7 and 8, the head coil 200 includes a fixed housing unit 210 and an adjustable-size housing unit 220, and a plurality of RF coil elements 240. The fixed housing unit 210 and the adjustable-size housing unit 220 are coupled to each other to provide a receiving space having a hemispherical shape in which the head of the patient 10 may be received.

A plurality of RF coil elements 241 are mounted in the fixed housing unit 210 (FIG. 8), and the fixed housing unit 210 covers the back part of the head of the patient 10. The fixed housing unit 210 may have a shape corresponding to a shape of the back part of the patient 10. The fixed housing unit 210 may extend to the neck of the patient 10. The fixed housing unit 210 may be formed of a rigid material or a flexible material.

Each of the plurality of RF coil elements 241 may be, for example, a circular, elliptical, or polygonal loop coil. The plurality of RF coil elements 241 may be arranged so that some overlap each other or are spaced apart from each other. A tuning capacitor or a decoupling capacitor may be provided on the RF coil elements 241. The RF coil elements 241 may operate using a multi-channel method in which RF signals are independently input or output by the RF coil controller 452 (see FIG. 13). For example, in the RF coil elements 241, circuits may be arranged in parallel and intensities and phases of RF signals that are input or output may be independently controlled. The RF coil elements 241 may be grouped and RF signals may be independently input or output according to groups.

The fixed housing unit 210 may be detachably mounted on the table 10 (see FIG. 1) on which the patient 10 lies by using a coupling device (not shown).

The adjustable-size housing unit 220 that receives the head of the patient 10 along with the fixed housing unit 210 includes a plurality of housing pieces 221 that extend from a plurality of points of a circumference of the fixed housing unit 210 and are bendably connected to one another and a fixing member 230 that is configured to fix the plurality of housing pieces 221.

The housing pieces 221 that are unfolded may form rows that extend from different points of the circumference of the fixed housing unit 210. Each of the housing pieces 221 may be formed of a rigid material or a flexible material. The housing pieces 221 may be formed to have a curved shape corresponding to a shape of the head or may be formed to have a plate-like shape. Although the housing pieces 221 are formed to have rectangular plate-like shapes in FIGS. 7 and 8, the present embodiment is not limited thereto. Alternatively, the housing pieces 221 may be formed to have circular, elliptical, or polygonal plate-like shapes, just to name some non-limiting possibilities. Alternatively, the housing pieces 221 may not need to have the same shape, and some may have rectangular plate-like shapes and others may have circular plate-like shapes.

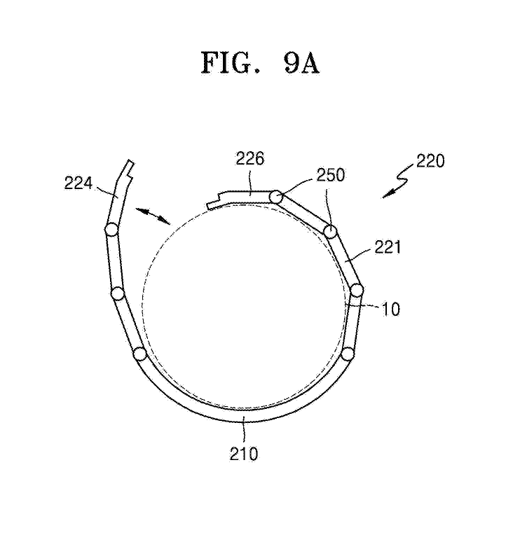

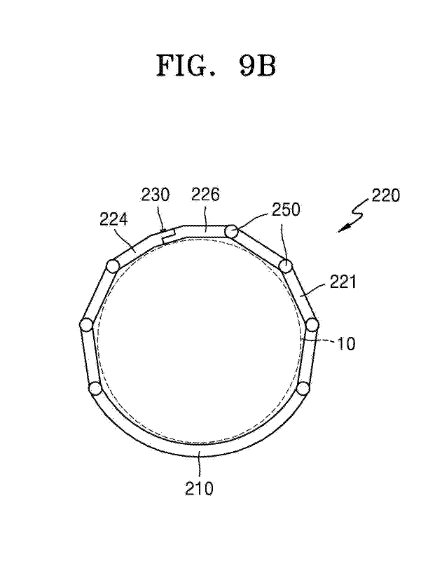

The housing pieces 221 may be connected to one another by using fasteners such as hinges 250 (see FIG. 9A) that be bent. Alternatively, the housing pieces 221 may be connected to one another by using a flexible material (e.g., rubber (natural, synthetic, plastic, etc.).

The housing pieces 221 include a mouth housing piece 222 that is located at a position corresponding to the mouth of the patient 10 and an eye housing piece 223 that is located at a position corresponding to the eyes of the patient 10. An opening 222a may be formed over the mouth in the mouth housing piece 222 to expose the mouth of the face to the outside. Likewise, an opening 223a may be formed over the eyes in the eye housing piece 223 to expose the eyes of the face to the outside. When imaging is performed for a long time, a hard housing structure has a small field of view, and thus may make the patient 10 feel enclosed and tired. However, since the openings 222a and 223a are respectively formed in the mouth housing piece 222 and the eye housing piece 223, a field of view may be increased, and thus the extent to which the patient 10 feels enclosed and tired may be minimized even when imaging is performed for a long time.

With reference to FIG. 8, one RF coil element 242, 243, or 244 is mounted on each of the housing pieces 221. Although two RF coil elements 244 are shown in FIG. 8, the RF coil elements may be provided in all of the housing pieces 221. The RF coil elements 242, 243, and 244 may be, for example, circular, elliptical, or polygonal loop coils. A tuning capacitor or a decoupling capacitor may be provided on the RF coil elements 242, 243, and 244. The RF coil elements 242, 243, and 244 may operate using a multi-channel method in which RF signals are independently input or output by the RF coil controller 452 (see FIG. 13). For example, in the RF coil elements 242, 243, and 244, circuits may be arranged in parallel and intensities and phases of RF signals that are input or output may be independently controlled. If necessary, the RF coil elements 242, 243, and 244 may be grouped and RF signals may be independently input or output according to groups.

Although one RF coil element 242, 243, or 244 is provided in each of the housing pieces 221, the present embodiment is not limited thereto. The plurality of RF coil elements 242, 243, and 244 may be disposed in each of the housing pieces 221. When the head coil 200 is mounted on the head of the patient 10, some of the housing pieces 221 of the adjustable-size housing unit 220 may overlap each other. Accordingly, the shielding member 330 (see FIGS. 11A and 11B) for shielding electromagnetic coupling between adjacent RF coil elements may be disposed on each of the RF coil elements 242, 243, and 244. The shielding member 330 will be explained below in detail with reference to FIGS. 11A, 11B, and 12.

Reference numerals 222, 223, 224, 225, 226, and 227 denote distal housing pieces located at ends of the rows that extend from different points of the circumference of the fixed housing unit 210 when the housing pieces 221 are unfolded. In the present embodiment, reference numerals 222 and 223 also denote the mouth housing piece and the eye housing piece.

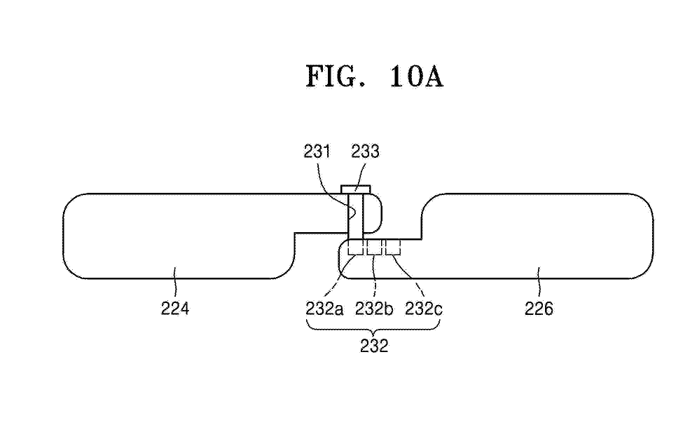

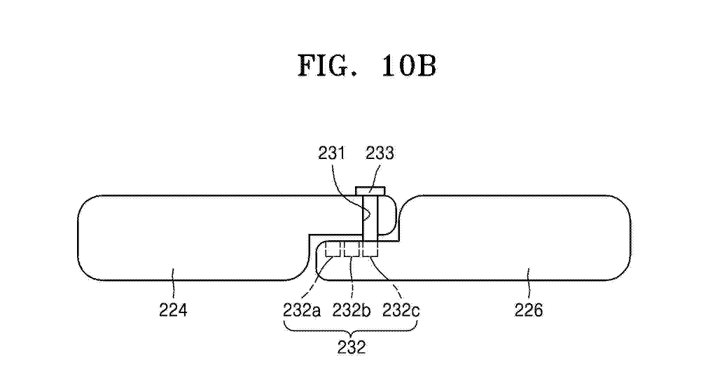

A first fixing sub-member 231 and a second fixing sub-member 232 for detachably fixing the distal housing pieces 222, 223, 224, 225, 226, and 227 are provided on the distal housing pieces 222, 223, 224, 225, 226, and 227. The first fixing sub-member 231 may be a hole into which, for example, a bolt 233 (see FIG. 10A) may be inserted, and the second fixing sub-member 232 may be a female screw engaged with the bolt 233. A plurality of female screw holes 232a, 232b, and 232c (see FIGS. 10A and 10B) may be formed in the second fixing sub-member 232 in an extension direction in which the housing pieces 221 extend to correspond to a size of the head. Alternatively, a plurality of holes may be formed in the first fixing sub-member 231 in the extension direction of the housing pieces 221 to correspond to a size of the head. The above structure of the fixing member 230 is and the present embodiment is not limited thereto. For example, the first fixing sub-member 231 may be formed as a long slot that extends in the extension direction of the housing pieces 221, and one female screw hole may be formed in the second fixing sub-member 232. Alternatively, the fixing member 230 may have a structure in which a protrusion is inserted into a groove or any of other well-known fasteners may be used.

For example, the plurality of housing pieces 221 are bendably connected at the bottom left side of the circumference of the fixed housing unit 210 to form a left row, and the mouth housing piece 222 is located at an end of the left row. Also, the plurality of housing pieces 221 are bendably connected at the bottom right side of the circumference of the fixed housing unit 210 to form a right row, and the distal housing piece 227 is located at an end of the right row. The first fixing sub-member 231 and the second fixing sub-member 232 for detachably fixing the mouth housing piece 222 and the distal housing piece 227 are respectively provided on the mouth housing piece 222 and the distal housing piece 227. As described below, when the head coil 200 is mounted on the head of the patient 10, the mouth housing piece 222 and the distal housing piece 227 are fixedly coupled to each other.

The distal housing pieces 224 and 226 at the top left and right sides of the circumference of the fixed housing unit 210 may be fixedly coupled to the distal housing piece 225 at the top side, and thus the first fixing sub-member 231 is provided on the distal housing piece 225 and the second fixing sub-member 232 is provided on the distal housing pieces 224 and 226. Since the distal housing pieces 224 and 226 at the top left and right sides may be fixedly coupled to each other, the first fixing sub-member 231 may be provided on one of the distal housing pieces 224 and 226 and the second fixing sub-member 232 may be provided on the remaining distal housing piece.

FIGS. 9A and 9B are views illustrating a process of mounting the head coil 200 onto the head of the patient 10. FIGS. 10A and 10B are views illustrating an example in which the head coil 200 of FIG. 8 is fixed by using the fixing member 230. In order to mount the head coil 200 on the head of the patient 10, when the head of the patient 10 is placed on the fixed housing unit 210, the plurality of housing pieces 221 of the adjustable-size housing unit 220 are bent to surround the head of the patient 10 and are fixed by using the fixing member 230 as shown in FIGS. 9A and 9B. When the plurality of housing pieces 221 are fixed by using the fixing member 230, the distal housing pieces 224 and 226 may partially overlap each other according to a size of the head of the patient 10.

Referring now to FIGS. 10A and 10B, fixed portions of the distal housing pieces 224 and 226 may be stepped so that the distal housing pieces 224 and 226 partially overlap each other and thus thicknesses of the fixed portions of the distal housing pieces 224 and 226 may be small. The plurality of female screw holes 232a, 232b, and 232c of the second fixing sub-member 232 are formed in an extension direction in which the housing pieces 221 extend to correspond to a size of the head. Accordingly, the bolt 233 may be inserted into one of the female screw holes 232a, 232b, and 232c according to an amount by which the distal housing pieces 224 and 226 overlap each other. When the head of the patient 10 is large, since the distal housing pieces 224 and 226 may overlap by a small amount, the bolt 233 is inserted into the female screw hole 232a that is an outermost hole from among the female screw holes 232a, 232b, and 232c as shown in FIG. 10A. When the head of the patient 10 is small, since the distal housing pieces 224 and 226 may overlap each other by a large amount, the bolt 233 is inserted into the female screw hole 232c that is an innermost hole from among the female screw holes 232a, 232b, and 232c as shown in FIG. 10B. Although three female screw holes 232a, 232b, and 232c are shown in FIGS. 10A and 10B, the present embodiment is not limited thereto and two female screw holes may be formed or four or more female screw holes may be formed.

As described above, a size of a receiving space of the head of the patient 10 that is formed by the fixed housing unit 210 and the adjustable-size housing unit 220 may be changed by adjusting a fixed position between the distal housing pieces 222, 223, 224, 225, 226, and 227 according to a size of the head of the patient 10. Since a size of the adjustable-size housing unit 220 may be adjusted according to a size of the head of each patient, the head coil 200 including the adjustable-size housing unit 220 may be understood as a reconfigurable head coil.

FIG. 11A is a plan view of an RF coil element 300 of a head coil according to another embodiment of the present disclosure. FIG. 11B is a cross-sectional view of the RF coil element 300 of FIG. 11A.

Referring now to FIGS. 11A and 11B, the RF coil element 300 includes an RF loop coil 320 having an octagonal shape (in this example) disposed on a basic material 310, and a shield loop coil 330 surrounding an outer surface of the RF loop coil 320. The RF coil element 300 of the present embodiment may be understood by the artisan as being any of the RF coil elements of the previous embodiments. For example, the RF coil element 300 may be any of the RF coil elements 140 and 170 of the embodiment of FIG. 1 through 6C or the RF coil elements 240 of the embodiment of FIGS. 7 through 10B. When the RF coil element 300 is one of the RF coil elements 240 of the embodiment of FIGS. 7 through 10B, the basic material 310 may be understood as one of the housing pieces 221.

The RF loop coil 320 and the shield loop coil 330 may be formed of a metal having high conductivity such as copper (Cu) or may be formed by coating a copper plate with silver (Ag) or gold (Au). A tuning capacitor for adjusting an RF signal impedance to be transmitted may be provided on the RF loop coil 320. A cable 350 for inputting/outputting an RF signal is connected to the RF loop coil 320.

The shield loop coil 330 may be located on the same plane as a plane on which the RF loop coil 320 is located, and may be disposed to have the same center as a center of the RF loop coil 320. The shield loop coil 330 may have the same shape as a shape of the RF loop coil 320. The shield loop coil 330 is electrically isolated from the RF loop coil 320.

A shape of the RF loop coil 320 shown in FIG. 11A is not limited to an octagonal shape, and may be, for example, a circular, elliptical, or polygonal shape. The shield loop coil 330 is a shielding member for shielding electromagnetic coupling with an adjacent RF loop coil 320. When the head coil 200 of the embodiment of FIGS. 7 through 10B is mounted on the head of the patient 10, some of the housing pieces 221 of the adjustable-size housing unit 220 may overlap each other, and thus the RF loop coils 320 in the housing pieces 221 may overlap each other. In this case, the shield loop coil 330 may shield electromagnetic coupling between the overlapping RF loop coils 320.

FIG. 12 is a plan view illustrating an arrangement in which two RF coil elements 300A and 300B overlap each other by a distance (that is, a gap) “d”. Table 1 shows a result obtained by measuring an S21 value by connecting the two RF coil elements 300A and 300B to both ports of a network analyzer when the two RF coil elements 300A and 300B overlap each other by the distance d as shown in FIG. 12. The S21 value is a transmission coefficient that is one of S parameters and may indicate whether decoupling is sufficient or not.

TABLE 1

| | Distance (mm) | | | | | | | | |
|---|---|---|---|---|---|---|---|---|---|
| | −10 | −5 | 0 | 5 | 7.5 | 10 | 12.5 | 15 | 20 |
| Default | −1.7 | −1.6 | −1.6 | −1.7 | −4.0 | −21 | −1.8 | −1.7 | −1.7 |
| (dB) | | | | | | | | | |
| RF | −11 | −8.8 | −6.3 | −5.6 | −7.3 | −11 | −43 | −21 | −7.2 |
| Shield | | | | | | | | | |
| (dB) | | | | | | | | | |

In Table 1, Default indicates a case where there is only the RF loop coil 320 without a shield loop coil. RF Shield indicates a case where there are both the RF loop coil 320 and the shield loop coil 330, that is, there is the RF coil element 300 of the present embodiment. When the distance d is a negative (−) value, it means that the two RF coil elements 300A and 300B are spaced apart from each other without overlapping each other.

As shown in Table 1, in Default, the S21 value is equal to or less than −11 dB when the distance d ranges from about 9 mm to about 11 mm. In RF Shield, the S21 value is equal to or less than −11 dB when the distance d ranges from about 10 mm to about 18 mm. In other words, a clearance that allows the S21 value to be equal to or less than −11 dB is 2 mm in Default whereas a clearance that allows the S21 value to be equal to or less than −11 dB is 8 mm in RF Shield. That is, the RF coil element 300 of the present embodiment may secure sufficient decoupling and may secure a clearance of 8 mm in which the two RF coil elements 300A and 300B may overlap each other. In the head coil 100 or 200 whose receiving space may vary according to a size of the head of each patient, some RF coil elements may overlap and an interval between the RF coil elements may be changed. In this case, since the RF coil element 300 of the present embodiment includes the shield loop coil 330 to increase a width of a clearance, even when an interval between some RF coil elements is changed, electromagnetic coupling may be shielded, thereby maintaining a high reception sensitivity and a high-quality output RF signal.

FIG. 13 is a view of the MRI apparatus 400 according to another embodiment. Referring now to FIG. 13, the MRI apparatus 400 of the present embodiment includes a cylindrical magnetic structure 410 and a computing device 450.

The cylindrical magnetic structure 410 includes a body RF coil 414, a gradient magnetic field coil 415, and a main magnet 416 in an inside-to-outside order. The body RF coil 414, the gradient magnetic field coil 415, and the main magnet 416 are mounted in a cylindrical gantry 419. An object (that is, a patient) that lies on a table 417 is moved into a bore 419a of the gantry 419, and then MRI is performed.

The body RF coil 414, the gradient magnetic field coil 415, and the main magnet 416 are connected to the computing device 450 and are driven and controlled. The computing device 450 may display an MRI image of the object or may be connected to a console (not shown) to which a piece signal of a user is input.

The body RF coil 414 of the MRI apparatus 400 may be independently driven and controlled by the RF coil controller 452 of the computing device 450 along with a head coil 412 mounted on the head of the object. Any of various local RF coils, instead of the head coil 412, may be used in the MRI apparatus 400 according to a body part (e.g., spine, shoulder, breast, torso, knee, or ankle).

The main magnet 416 for generating a main magnetic field for magnetizing an atomic nucleus of an element that exhibits an MR phenomenon, such as hydrogen, phosphorus, sodium, or carbon, among elements distributed in a human body may be a superconductive electromagnet or a permanent magnet.

The gradient magnetic coil 415 generates a spatially linear gradient magnetic field in order to capture an MR image. In general, three gradient magnetic file coils for respectively forming gradient magnetic fields along x-, y-, and z axes are used to capture an MR image. The gradient magnetic field coil 415 spatially controls a rotating frequency or a phase of a magnetization vector when the magnetization vector rotates in a horizontal plane, in order to express an MRI image in a spatial frequency region, that is, a k-region.

A magnetization vector has to be arranged in a horizontal plane in order to produce a MR image signal. To this end, the body RF coil 414 for generating an RF magnetic field having a Larmor frequency as a center frequency is required. The body RF coil 414 to which an RF current in a Larmor frequency band is applied generates a rotating magnetic field that rotates at a Larmor frequency. Due to the rotating magnetic field, the magnetization vector resonates, that is, nuclear magnetic resonance occurs, and thus the magnetization vector lies in the horizontal plane. Once the magnetization vector lies in the horizontal plane, the magnetization vector that rotates at the Larmor frequency in the horizontal plane generates an electromotive force in the body RF coil 414 or the head coil 412 according to Faraday's Law. When the electromotive force, that is, a received RF signal, is amplified by using a high frequency amplifier and then is demodulated into a sine wave having the Larmor frequency, an MR signal in a base band may be obtained. The MR signal in the base band is transmitted to the computing device 450 and is quantized by using an image processor 451, to obtain an MR image.

A process of generating an MR image in the MRI apparatus 400 has been described briefly. A detailed explanation of a process of generating an MR image is well known to one of ordinary skill in the art, and thus will not be given.

The body RF coil 414 provided in the gantry 419 of the MRI apparatus 400 may be used to capture an MR image of the whole body of the object. In contrast, in order to capture of an MR image of a body part of the object such as head, breast, or leg, a local RF coil (e.g., the head coil 412) provided on the body part of the object may be used. The local RF coil such as the head coil 412 is an independent device separately provided outside the gantry 419 and may be moved to be mounted on the body part of the object whose MR image is to be obtained. The head coil 412 may be any of the head coils 100 and 200 of the previous embodiments. As described above, in the head coil 412, since RF coil elements are closely fitted around the head irrespective of a size (volume) of the head of each patient by changing or modifying a part of a housing, the MRI apparatus 400 may obtain a high-quality image. Also, since the head coil 412 has a structure with a large field of view, even when the MRI apparatus 400 performs imaging for a long time, the extent to which the object (patient) feels enclosed and tired may be minimized.

While one or more embodiments have been described with reference to the figures, it will be understood by those of ordinary skill in the art that various changes in form and details may be made therein without departing from the spirit and scope as defined by the following claims.

What is claimed is:

1. A head coil for a magnetic resonance imaging (MRI) apparatus, comprising:
a plurality of radio frequency (RF) coil elements;
a first housing having a first housing surface in which one or more of the plurality of RF coil elements is embedded therein, and the first housing having an adjustable receiving space that corresponds to any of different head sizes of patients;
a second housing selected from a plurality of second housings having a structure configured to receive a head of a patient along with the first housing and having a second housing surface in which another one or more of the plurality of RF coil elements is embedded therein, and having one receiving space selected from among the plurality of second housings with respective receiving spaces of different sizes that correspond to different head sizes of patients; and
a coupling unit that detachably couples any one of the plurality of the second housings to the first housing.

2. The head coil for the MRI apparatus of claim 1, wherein the first housing is detachably mounted on a support that supports the patient.

3. The head coil for the MRI apparatus of claim 2, wherein the first housing covers a back part of the head of the patient, and the second housing covers at least a front part of the head of the patient.

4. The head coil for the MRI apparatus of claim 1, wherein the first housing comprises a plurality of housing pieces, and a fixing member configured to attach the plurality of housing pieces and to vary the receiving space by adjusting a fixed position between the plurality of housing pieces.

5. The head coil for the MRI apparatus of claim 1, wherein a shielding member for shielding electromagnetic coupling between adjacent RF coil elements is arranged on each of the plurality of RF coil elements.

6. The head coil for the MRI apparatus of claim 5, wherein each of the plurality of RF coil elements comprises an RF loop coil, and the shielding member comprises a shield loop coil surrounding an outer surface of the RF loop coil.

7. The head coil for the MRI apparatus of claim 6, wherein the shield loop coil is arranged on the same plane as the RF loop coil.

8. The head coil for the MRI apparatus of claim 7, wherein the shield loop coil is arranged to have the same center as a center of the RF loop coil.

9. A head coil for a magnetic resonance imaging (MRI) apparatus, comprising:
a plurality of radio frequency (RF) coil elements;
a fixed housing unit having a structure in which at least one or more of a plurality of RF coil elements are embedded therein to cover a back part of a head of a patient;
an adjustable-size housing unit configured to receive the head of the patient along with the fixed housing unit, in which the adjustable-size housing unit having embedded therein another one or more of the plurality of RF coil elements, and comprising a plurality of housing pieces that extend from a plurality of points of a circumference of the fixed housing unit and are bendably connected to one another; and
a fixing member configured to detachably fix a first and a second housing piece of the plurality of housing pieces respectively located at ends of the fixing member extending from a first point and a second point of the circumference of the fixed housing unit.

10. The head coil for the MRI apparatus of claim 9, wherein the plurality of housing pieces are connected to one another with hinges.

11. The head coil for the MRI apparatus of claim 9, wherein the fixing member includes a variably-sized receiving space formed by the fixed housing unit and the adjustable-size housing unit that receives a head of the patient by adjusting a fixed position between the first housing piece and the second housing piece at respective ends of the fixing member to correspond to respective head sizes of patients.

12. The head coil for the MRI apparatus of claim 11, wherein the first housing piece and the second housing piece at respective ends of the fixing member are coupled to each other to partially overlap each other.

13. The head coil for the MRI apparatus of claim 13, wherein the adjustable-size housing unit comprises an eye housing piece located over the eyes of the patient and a mouth housing piece located over the mouth of the patient, and the eye housing piece and the mouth housing piece have openings through which the eyes and the mouth of the patient are exposed to the outside.

14. The head coil for the MRI apparatus of claim 13, wherein the eye housing piece and the mouth housing piece respectively include an eye RF coil and a mouth RF coil, and each of the eye RF coil and the mouth RF coil is one of a circular, elliptical, or polygonal loop coil.

15. The head coil for the MRI apparatus of claim 9, wherein a shielding member for shielding electromagnetic coupling between adjacent RF coil elements is provided on each of the plurality of RF coil elements.

16. The head coil for the MRI apparatus of claim 15, wherein each of the plurality of RF coil elements comprises an RF loop coil, and the shielding member is a shield loop coil surrounding at least part of an outer surface of the RF loop coil.

17. The head coil for the MRI apparatus of claim 16, wherein the shield loop coil is located on the same plane as the RF loop coil.

18. The head coil for the MRI apparatus of claim 16, wherein the shield loop coil is arranged to have the same center as the RF loop coil.

19. The head coil for the MRI apparatus of claim 15, wherein as an inner receiving space of the adjustable-size housing unit is changed, a distance between at least one of the plurality of RF coil elements in the adjustable-size housing unit is changed or an amount by which at least some of the plurality of RF coil elements overlap each other is changed.

20. A magnetic resonance imaging (MRI) apparatus comprising:
a gantry comprising an imaging space to which a magnetic field for obtaining a magnetic resonance (MR) image is applied;
a main magnet module arranged in the gantry and configured to apply a main magnetic field;
a gradient coil module arranged in the gantry and configured to apply a gradient magnetic field;
a main body radio frequency (RF) coil module arranged in the gantry; and
a head coil configured to cover at least a part of the head of a patient,
wherein the head coil comprises:
a plurality of RF coil elements;
a first housing having a first housing surface in which one or more of the plurality of RF coil elements is embedded therein, and the first housing having an adjustable receiving space that corresponds to any of different heads sizes of patients;
a second housing having a structure configured to receive the head of a patient along with the first housing and having a second housing surface in which another one or more of the plurality of RF coil elements is embedded therein, and having one receiving space selected from among a plurality of second housings with respective receiving spaces of different sizes that correspond to different head sizes of patients; and
a coupling device that detachably couples to any one of the plurality of the second housings to the first housing.
